(19) United States
(12) Patent Application Publication
(10) Pub. No.: US 2026/0069230 A1
(43) Pub. Date: Mar. 12, 2026

(54) SPINAL FRACTURE DETECTION

(71) Applicant: KONINKLIJKE PHILIPS N.V., EINDHOVEN (NL)

(72) Inventors: ALENA-KATHRIN GOLLA, QUICKBORN (DE); CRISTIAN LORENZ, HAMBURG (DE); CHRISTIAN BUERGER, HAMBURG (DE); TANJA LOSSAU, HAMBURG (DE); TOBIAS KLINDER, UELZEN (DE)

(21) Appl. No.: 19/107,650

(22) PCT Filed: Aug. 31, 2023
(86) PCT No.: PCT/EP2023/073860

§ 371 Date: Feb. 28, 2025

Related U.S. Application Data
(60) Provisional application No. 63/402,983, filed on Sep. 1, 2022.
(60) Provisional application No. 63/451,656, filed on Mar. 13, 2023.

(51) Int. Cl. A61B 6/50; A61B 6/00; A61B 6/03; G06T 7/00; G06T 7/10; G06T 7/73

(52) U.S. Cl. CPC A61B 6/505; A61B 6/032; A61B 6/5217; G06T 7/0012; G06T 7/10; G06T 7/73; G06T 2200/04; G06T 2207/10081; G06T 2207/20076; G06T 2207/20084; G06T 2207/30012

(57) ABSTRACT

Three-dimensional image data that includes at least a portion of a spine of a subject is received. The spine of the subject is identified Retrieve in the three-dimensional image data, and a spline approximating a local curvature along the spine is defined. Multiple volumes of interest (VOIs) are defined, each VOI containing at least a portion of a vertebra of the spine of the subject. Each VOI is defined relative to an adjacent segment of the spline. A fracture in at least one of the VOIs is identified.

### FIELD

The present invention generally relates to systems and methods for spine fracture detection using machine learning. In particular, the invention relates to spine fracture detection using convolutional neural networks (CNNs) on spinal canal aligned volumes of interest (VOIs).

### BACKGROUND

In emergency department treatments, such as in the context of triage for trauma, it is often necessary to quickly assess all injuries, as well as an overall patient condition. Ideally, such an assessment would be as complete and as quick as possible. Usage of head to pelvis, or even head to toe, 3D computed tomography (CT) scans is widely established in order to support such a quick assessment.

Among potential injuries, identifying spinal injuries, such as fractures, is particularly important. Unstable fractures often require rapid surgical intervention and lead to subsequent injuries during hospital care due to transportation and handling of a patient. Consequently, cervical spine fractures in particular must be identified or ruled out as soon as possible in an unclear poly-trauma situation.

In existing methods, such CT scans are not easily used to detect spinal fractures. Because manual assessment of the spine is time consuming, in some automated methods based on imaging, such as CT scans, first assistance systems classify individual vertebrae as either fractured or not. However, it is not always possible to determine what led the method to indicate the presence of a fracture and, as such, it is not always possible to confirm the presence of such a fracture.

For example, in some fracture detection systems, a technician may be informed whether a given vertebra is broken or not. However, such systems are typically whole vertebra fracture classification systems, and information about the location of the fracture may not be provided, or may be provided only in the form of compressed previews of network decision maps. Accordingly, a positive indication from an automated fracture detection method may be difficult to confirm as either a fracture or a false positive.

As noted above, manual detection of spinal fractures is a lengthy procedure, and overlooked fractures can have serious health consequences for a patient. Failed detection of unstable fractures can lead to spinal cord injuries. Accordingly, a false negative identified by an automated fracture detection method, or an insufficiently thorough manual examination by a doctor, may have severe consequences. Similarly, incorrectly identifying the output of an automated method as a false positive has severe consequences.

Therefore, there is a need for a spinal fracture detection system and method in which a more precise fracture detection can be provided, and in which such a fracture detection can highlight a specific location of an assumed fracture. There is a further need for such a system that can indicate a confidence level, such as a confidence score, associated with identified fractures.

### SUMMARY

A method is provided for detecting spinal fractures. According to one embodiment of the invention, the method includes receiving three-dimensional image data that includes at least a portion of a spine of a subject.

According to one embodiment, the method proceeds with identifying the spine of the subject in the three-dimensional image data and defining a spline approximating a local curvature along the spine of the subject.

According to one embodiment, the method then proceeds to define a plurality of volumes of interest (VOIs), each VOI containing at least a portion of a vertebra of the spine of the subject. Each VOI is defined relative to an adjacent segment of the spline.

According to one embodiment, the method then identifies a fracture in at least one of the VOIs.

In some embodiments, the identifying of the spine is by a convolutional neural network (CNN) trained for segmenting the spine. In some such embodiments, the CNN is a foveal net applied to a CT image.

In some embodiments, each VOI of the plurality of VOIs has a corresponding center point sampled at a location defined relative to the spline. The center points of the plurality of VOIs are then sampled at regular intervals along the spline. In some such embodiments, center points for adjacent VOIs are located so as to generate overlapping VOIs.

In some embodiments, each VOI is formed about the center point and is oriented based on a tangent of the spline adjacent the corresponding center point. In some such embodiments, after defining each VOI of the plurality of VOIs, each VOI is extracted and resampled from the three-dimensional image data to a target resolution.

In some embodiments, the identification of a fracture is by applying a CNN to each VOI of the plurality of VOIs. In some such embodiments, the output of the CNN, when applied to a VOI of the plurality of VOIs, is a probability map identifying likely fractures within the corresponding VOI.

In some such embodiments, the plurality of VOIs is defined such that adjacent VOIs overlap, so as to generate multiple predictions for at least some equivalent voxels occurring in multiple VOIs. All predictions corresponding to a particular location are then aggregated into a final probability map.

In some embodiments, the method further includes generating a final probability map from the probability maps associated with individual VOIs. The final probability map then comprises a collation of the VOI probability maps into a coherent representation of the three-dimensional image data. In some such embodiments, the method further includes generating binary predictions based on the final probability map and filtering fracture candidates based on a relationship between a candidate location and the spine of the subject.

In some embodiments, each VOI of the plurality of VOIs includes a portion of the spine within the corresponding volume.

In some embodiments, the spline approximates a centerline of a spinal canal for the spine.

In some embodiments, a size for a first VOI of the plurality of VOIs is selected based on an adjacent first location along the spine. A size for a second VOI of the plurality of VOIs is then selected based on an adjacent second location along the spine, and the size for the first VOI is different than the size for the second VOI.

In some such embodiments, the size for the first VOI is based on a size of a vertebra structure expected at the first location along the spine.

In some embodiments, the method further includes locating fractures identified in a representation of the three-dimensional image data and displaying identified fractures in the context of the spine of the subject.

### BRIEF DESCRIPTION OF THE DRAWINGS

FIG. 1 is a schematic diagram of a system according to one embodiment of the present invention.

FIG. 2 illustrates an exemplary imaging device according to one embodiment of the present invention.

FIGS. 3A-E illustrate the segmentation of a spine in image data in accordance with the methods disclosed.

FIG. 4 illustrates the creation of a spine mask in accordance with the methods disclosed.

FIG. 5 illustrates the identification of volumes of interest (VOIs) in accordance with the methods disclosed.

FIG. 6 illustrates the extracted VOIs identified in FIG. 5.

FIG. 7 is a flowchart illustrating a method for spinal fracture detection in accordance with one embodiment of the present invention.

FIGS. 8A-B illustrate fractures identified in the context of image data.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present disclosure. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the disclosure expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the disclosure presented solely for illustrative purposes by reference to the accompanying drawings to advise one of ordinary skill in the art of the advantages and construction of the disclosure. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality.

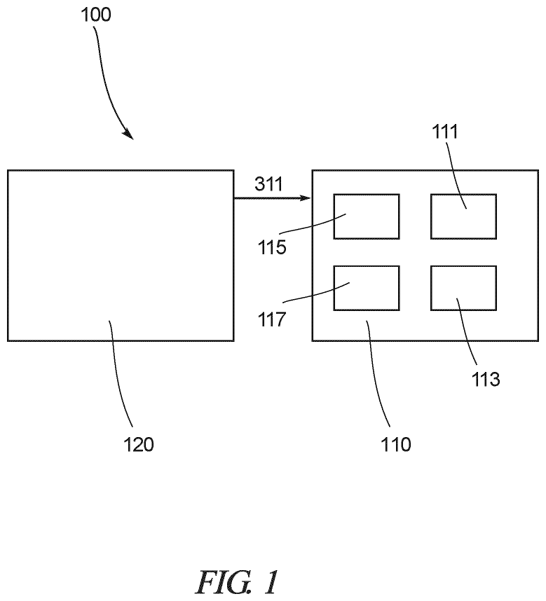

FIG. 1 is a schematic diagram of a system 100 according to one embodiment of the present invention. As shown, the system 100 typically includes a processing device 110 and an imaging device 120.

The processing device 110 may apply processing routines to images (image data) or projections (projection data) received from the imaging device 120. The processing device 110 may include a memory 113 and processor circuitry 111. The memory 113 may store a plurality of instructions. The processor circuitry 111 may couple to the memory 113 and may be configured to execute the instructions. The instructions stored in the memory 113 may comprise processing routines, as well as data associated with processing routines, such as machine learning algorithms, and various filters for processing images.

The processing device 110 may further include an input 115 and an output 117. The input 115 may receive information, such as three-dimensional image data or projection data, from the imaging device 120. The output 117 may output information, such as filtered images, or converted two-dimensional images, to a user or a user interface device. The output may include a monitor or display.

In some embodiments, the processing device 110 may relate to the imaging device 120 directly. In alternate embodiments, the processing device 110 may be distinct from the imaging device 120, such that the processing device 110 receives images or projection data for processing by way of a network or other interface at the input 115.

In some embodiments, the imaging device 120 may include a data processing device and a spectral CT scanning unit for generating CT spectral data when scanning an object (e.g., a patient). In some embodiments, the imaging device 120 may be a conventional CT scanning unit configured for generating scans.

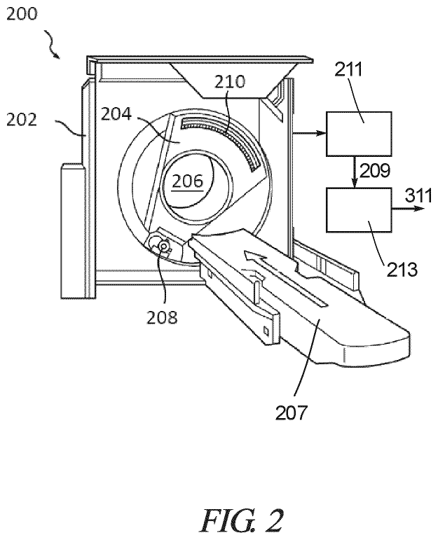

FIG. 2 illustrates an exemplary imaging device 200 according to one embodiment of the present disclosure. It will be understood that while a CT imaging device 200 is shown, and the following discussion is generally in the context of CT images, similar methods may be applied in the context of other imaging devices, and images to which these methods may be applied may be acquired in a wide variety of ways.

In an imaging device 200 in accordance with embodiments of the present invention, the CT scanning unit may be adapted for performing axial scans and/or a helical scan of an object in order to generate the CT projection data. In an imaging device 200 in accordance with embodiments of the present invention, the CT scanning unit may comprise an energy-resolving photon counting detector or a spectral dual-layer image detector. Spectral content may be acquired using other detector setups as well. The CT scanning unit may include a radiation source that emits radiation for traversing the object when acquiring the projection data.

In the example shown in FIG. 2, the CT scanning unit 200, e.g. the Computed Tomography (CT) scanner, may include a stationary gantry 202 and a rotating gantry 204, which may be rotatably supported by the stationary gantry 202. The rotating gantry 204 may rotate about a longitudinal axis around an examination region 206 for the object when acquiring the projection data. The CT scanning unit 200 may include a support 207 to support the patient in the examination region 206 and configured to pass the patient through the examination region during the imaging process.

The CT scanning unit 200 may include a radiation source 208, such as an X-ray tube, which may be supported by and configured to rotate with the rotating gantry 204. The radiation source 208 may include an anode and a cathode. A source voltage applied across the anode and the cathode may accelerate electrons from the cathode to the anode. The electron flow may provide a current flow from the cathode to the anode to produce radiation for traversing the examination region 206.

The CT scanning unit 200 may comprise a detector 210. The detector 210 may subtend an angular arc opposite the examination region 206 relative to the radiation source 208. The detector 210 may include a one-or two-dimensional array of pixels, such as direct or indirect conversion detector pixels. The detector 210 may be adapted for detecting radiation traversing the examination region 206 and for generating a signal indicative of an energy thereof.

The CT scanning unit 200 may include generators 211 and 213. The generator 211 may generate tomographic projection data 209 based on the signal from the detector 210. The generator 213 may receive the projection data 209 and, in some embodiments, generate three-dimensional CT image data 311 of the object based on the projection data 209. In some embodiments, the projection data 209 may be provided to the input 115 of the processing device 110, while in other embodiments the three-dimensional CT image data 311 are provided to the input of the processing device.

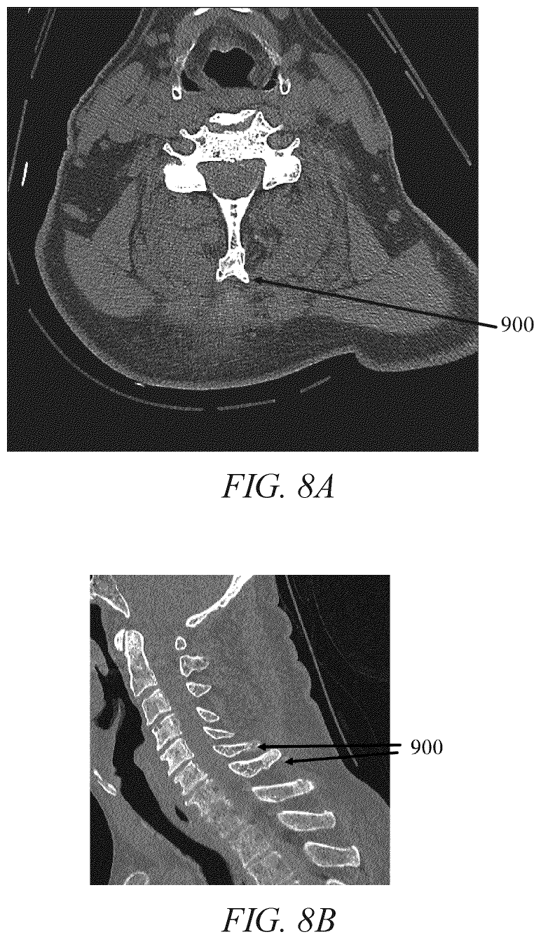

In the context of three-dimensional CT images 311, a spine fracture detection algorithm may formulate fracture detection as a segmentation task, rather than a classification task. Accordingly, the method may sample volumes of interest (VOIs) and for each voxel of a given VOI, the method may attempt to determine if the corresponding voxel of a vertebra is part of a fracture or not. This approach further allows for the generation of renderings with a voxel-wise highlighted or colored indication of an exact fracture location, as shown below in FIGS. 8A-8B. By highlighting a specific location, the method allows a clinician to efficiently check if they agree with the algorithm. Such an approach also builds trust, since the clinician can better understand the basis for the fracture/no fracture classification decision made by the system.

In order to evaluate the spine in the context of the segmentation task, the spine is segmented, and then a spinal canal centerline is extracted. Once the centerline is extracted, VOIs may be sampled along the centerline, and such VOIs may be oriented based on a local angle of the centerline. Once the VOIs are sampled, they may be evaluated using learning algorithms, such as CNNs, in terms of the underlying voxels.

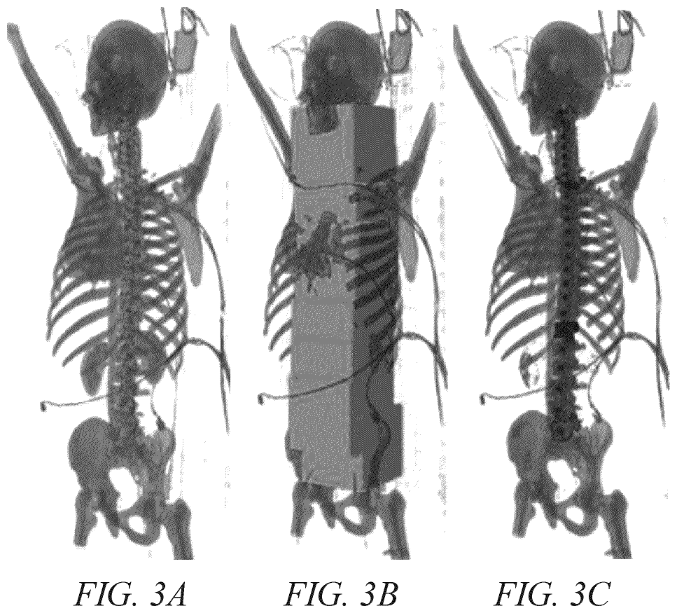

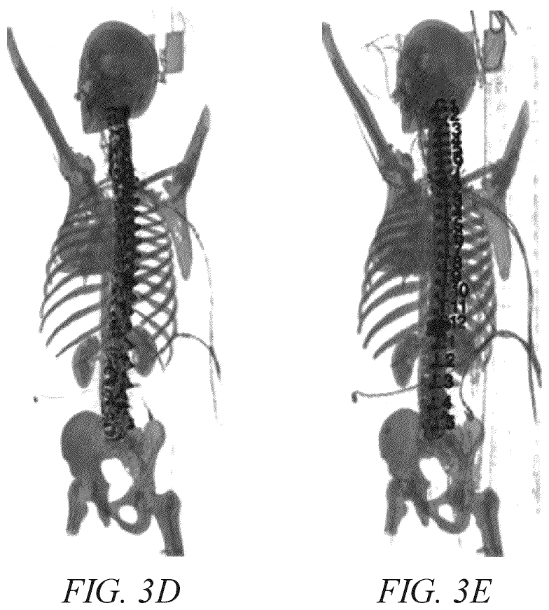

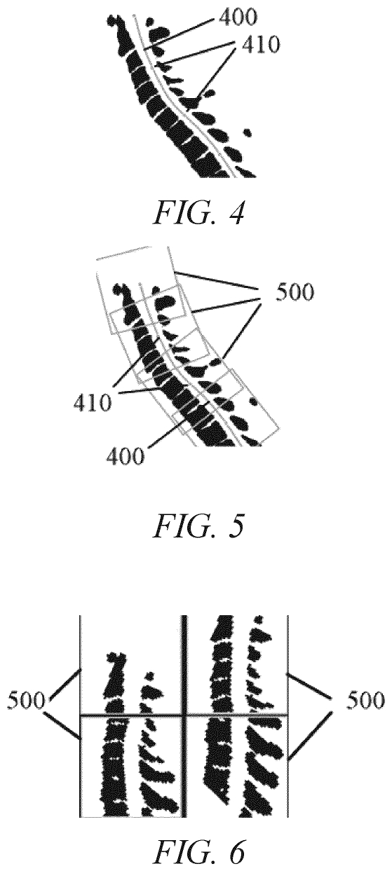

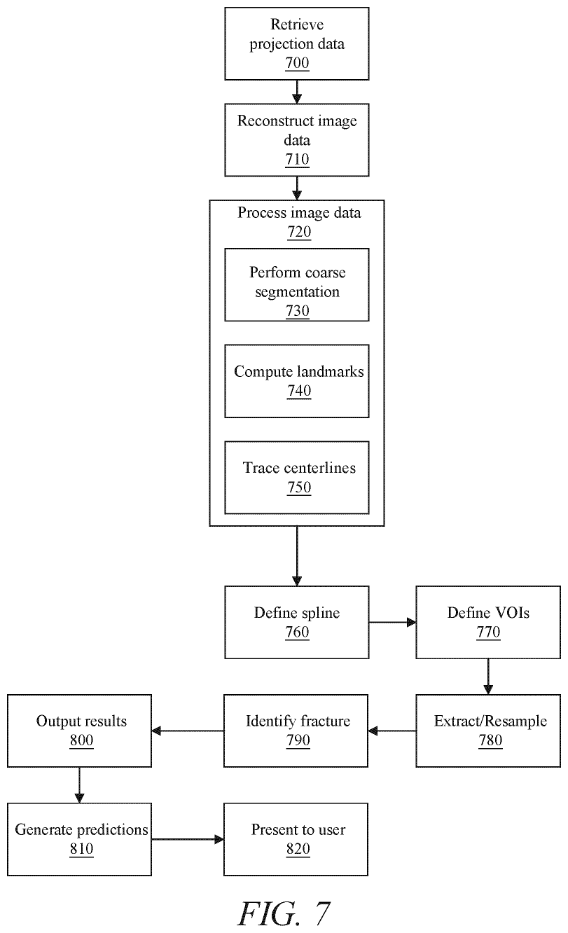

FIGS. 3A-E illustrate the segmentation of a spine in the image data in accordance with the embodiments disclosed herein. FIG. 4 illustrates the creation of a spine mask in accordance with the embodiments disclosed herein. FIG. 5 illustrates the identification of volumes of interest (VOIs) 500 in accordance with the embodiments disclosed herein. FIG. 6 illustrates the extracted VOIs 500 identified in FIG. 5. FIG. 7 is a flowchart illustrating spinal fracture detection in accordance with one embodiment of the present invention.

As shown in FIG. 7, the method for spinal fracture detection typically first retrieves data (700). The retrieved data is projection data acquired from a plurality of angles about a central axis.

Accordingly, in the context of the imaging device 200 of FIG. 2, the subject may be a patient on the support 207, and the central axis may be an axis passing through the examination region. The rotating gantry 204 rotates about the central axis of the subject, thereby acquiring the projection data from various angles.

Once acquired, the projection data 311 is reconstructed (710) as a three-dimensional image data 300 in preparation for further processing in the image domain.

The data (retrieved at 700) includes at least a portion of a spine of a subject being analyzed. The processing (at 720) then includes the identification of the spine of the subject in the three-dimensional images. Such identification may be done in a number of ways, but one approach is shown in FIGS. 3A-3E.

The approach relies on a segmentation process for segmenting the spine in the three-dimensional image data. Accordingly, the method may first apply a course segmentation (730) to globally separate the spine as a single class from the rest of the image. This course segmentation is shown in FIG. 3A. Such course segmentation may be by applying a learning algorithm, such as a convolutional neural network (CNN) to the entire field of view (FOV) contained in the imaging data. The CNN may be, for example, a foveal net (FNET) applied to a CT image. The FNET may be applied to the CT image with an isotropic resampling of 5.0 mm.

As shown in FIGS. 3B, 3C, and 3D, following the course segmentation of the spine in the entire FOV, multiple bounding boxes may be placed around the spine to locally refine the segmentation. Within each bounding box, a further learning algorithm, such as a CNN, may be applied locally. In the embodiment shown, a UNET may be applied within each bounding box with an isotropic resampling of 2.0 mm. This finer segmentation may then generate a multi-class output, resulting in segmenting an intermediate vertebra class, a spinal canal class, and a landmark class representing the center of gravity within each vertebra body, along with multiple key vertebra structures.

FIG. 3B shows patch sampling of the initial course segmentation to allow for further segmentation. FIG. 3C then shows further segmentation into different class, allowing the labeling of multiple key vertebra structures, such as C2, T1, T-Last, and L-Last. Once these landmarks are identified and labelled, the method can proceed to count through the sorted landmark class, allowing the computation of the labelled landmarks of all vertebra body centers visible in the field of view (740), as shown in FIG. 3D.

Once all landmarks are identified, a centerline is traced (750) based on the landmarks previously identified at 740. The centerline typically corresponds to the spinal canal class of the refined segmentation, and comprises a set of points along the centerline.

As shown in FIG. 4, once the set of points 410 is traced, a spline 400 is defined (760) approximating local curvature along the spine of the subject. In the embodiment shown, such a spline is based on the points 410 defined as the centerline (750), and therefore corresponds directly to the identified spinal canal. Accordingly, in the spine mask shown in FIG. 4, the spinal canal centerline 400 is shown as the generated spline, and the sampled points 410 are shown linked by the spline. However, it will be understood that the spline may be defined in other ways as well, so long as it approximates local curvature.

Once the spline 400 is defined, multiple volumes of interest (VOIs) 500 (770) are defined as shown in FIG. 5. Each such VOI contains at least a portion of a vertebra of the spine of the subject, and each VOI is defined relative to an adjacent segment of the spline 400.

In the embodiment shown in the figures, each VOI 500 is centered on a point 410 sampled as part of the definition of the centerline (750). The points 410 may then be sampled at regular intervals along the spline 400. In some embodiments, sampled points 410 used to define the spline are used as shown in FIG. 5. In alternative embodiments, a set of points may be defined along the spline to proactively locate the center points of each VOI.

As shown in FIG. 5, when defining each VOI 500, the corresponding VOI is formed about the corresponding center point 410 and may be oriented based on a tangent of the spline 400 adjacent the corresponding center point. In this way, the orientation of each VOI may correspond to the local curvature of the spine, which may improve performance of algorithms applied to the VOI. Accordingly, the orientation step reduces the geometrical variability that the classification network, such as the CNN discussed below, needs to learn. This increases robustness and reduces the size of the training set required.

In the embodiment shown in the figures, the VOIs 500 may be defined so that they overlap. Where points 410 are sampled at regular intervals along the spline 400, such points may be located such that they are closer than a width of the VOI. For example, in the embodiment shown in FIG. 5, each VOI 500 center point 410 is sampled with a distance of 20 mm on the spline 400. Each VOI may then have a size of, for example, 128×128×128 voxels in a processing resolution of 0.7×0.7×0.7. Accordingly, the sampling distance is chosen to generate overlapping VOIs 500. In this way, once fracture detection algorithms are applied to each VOI, overlapping sections of VOIs may be provided with multiple distinct results associated with each VOI. Each vertebra is then processed in multiple views both in training and in inference. The VOIs are positioned such that the complete sampled set covers the whole spinal column but not much more, which may speed up future processing steps.

In some embodiments, each VOI may be deliberately defined such that each VOI includes a portion of the spine within the corresponding volume. Such an approach assists algorithms to be later applied by allowing the spinal segment to be used for orientation purposes. In some embodiments, VOIs may be utilized without replicating the adjacent spinal cord segment.

In some embodiments, each VOI 500 may be identical in size. This is shown, for example, in FIG. 5 and is discussed above. However, in some embodiments, different VOIs may be sized differently. For example, a size for a first VOI may be selected based on an adjacent first location along the spine, and a size for a second VOI may be selected based on an adjacent second location along the spine. The size for the first VOI may then be different than the size for the second VOI. As noted above, the spinal segmentation may provide a class segmentation, as shown in FIG. 3C. Such a class segmentation may locate each VOI relative to different types of vertebrae, and as such, each VOI may be sized based on a size of local vertebrae. For example, lumbar vertebrae are larger than cervical vertebrae, and therefore require a larger VOI. Accordingly, the size of the first VOI may be based on a size of a vertebra structure expected at the first location, while the size of the second VOI may be based on a size of a vertebra structure expected at the second location. Similarly, the distance between the center points sampled 410 may, in some embodiments, be varied based on the size of the local vertebra structure.

In some embodiments, after defining each VOI 500, each VOI is then extracted and resampled (780) from the three-dimensional images to a target resolution. The target resolution may be a resolution in which algorithms designed for fracture detection operate. This may be different than an initial target resolution used for the segmentation of the spine (730). As noted above, the orientation of the VOI may be based on the local curvature of the spline 400. Accordingly, in resampling the VOI, the volume may be rotated, so that all processing may be better aligned with the local tangent vector of the spline 400.

FIG. 6 shows the extracted VOIs 500 as identified in FIG. 5. As shown, each VOI 500 has now been resampled and oriented so that they are aligned with the tangent of the spline 410 at the center of the VOI. Typically, other than resampling, no further reformatting is applied, so as to avoid introducing any distortions.

Following the resampling of each VOI (780), a fracture is identified (790) in at least one of the VOIs 500. The identification of a fracture may be by applying a learning algorithm, such as a convolutional neural network (CNN) to each VOI. The results of CNN (800) are output, such that any identified fractures may be evaluated. The CNN architecture may be a UNET used for segmentation, but landmark detection or bounding box regression are contemplated as well.

As noted above, the VOIs may be defined so as to include the spine as a support structure, and as such, the network may be trained to simultaneously segment the spine and the fractures. Including the spine as a support structure in training and in processing may achieve higher performance than only segmenting fractures. As such, some embodiments include deliberately including the spine in the VOIs. During network training, case weighting based on fracture occurrence and types may be applied as well as an adapted sampling to ensure a suitable ratio between spine and fracture samples.

The output of the CNN (800) when applied to a VOI 500 of the multiple VOIs may be a probability map identifying likely fractures within the corresponding VOI. Such probability maps may then be resampled back into the original image space, such as into the three-dimensional images. As noted above, the VOIs 500 may be defined such that adjacent VOIs overlap. This generates multiple predictions for at least some equivalent voxels contained in multiple VOIs. All predictions corresponding to a particular location are then aggregated into a final probability map. Accordingly, in some embodiments, following the output of the CNN (800), a final probability map is generated from the probability maps associated with individual VOIs. The final probability map then comprises the collation of the VOI probability maps into a coherent representation of the three-dimensional image data. Such collation may be, for example, through a maximum operation.

Once the probability map is complete, post processing may be required to extract final fracture candidates from the maps. Binary predictions (810) are generated based on the final probability map, such as by using argmax operations or by thresholding the final fracture probability maps. Fracture candidates may then be filtered based on a relationship between a candidate location and the spine of the subject, for example, to exclude potential false positives outside the spine. The resulting predictions may be masked with the dilated spine mask shown in FIG. 3E.

Candidates may then be identified via connected component analysis of the binary predictions. Components can be filtered by their size to exclude small fragments. Finally, a confidence score can be calculated for each candidate based on the fracture probability.

Once predictions are made (810), such predicted fractures may be identified and located in a representation of the three-dimensional image data. Accordingly, the images, including the predicted fractures, may be presented (820) to a user in the context of the spine of the subject.

FIGS. 8A-B illustrate fractures identified in the context of image data. Such figures show how fracture candidates 900 may be visualized in the original data. As shown, fracture candidates 900 may be highlighted in the context of an otherwise original image.

The embodiments of the present invention can be implemented in various contexts, including CT consoles associated with specific CT scanners, as well as medical viewing workstations, PACS systems, or cloud-based systems.

A receiving operating characteristic (ROC) curve may be generated for validating the fracture detection described. In some embodiments, such a curve may be an ROC curve in a 4-fold cross-validation setting, using 200 training cases, with several fracture candidates identified per case. A fracture detection sensitivity of 87 percent could be reached when allowing for 4 false positives per case. In any event, a confidence score may be utilized to indicate confidence in any particular fracture candidate. Such a confidence score may be used as a thresholding metric, which can in turn be used to select a sensitivity of the embodiments described herein. A more sensitive implementation may result in additional false positives identified.

The embodiments according to the present disclosure may be implemented on a computer as a computer implemented method, or in dedicated hardware, or in a combination of both. Executable code for a method according to the present disclosure may be stored on a computer program product. Examples of computer program products include memory devices, optical storage devices, integrated circuits, servers, online software, etc. Preferably, the computer program product may include non-transitory program code stored on a computer readable medium for performing a method according to the present disclosure when the program product is executed on a computer. In an embodiment, the computer program may include computer program code adapted to perform all the steps of a method according to the present disclosure when the computer program is run on a computer. The computer program may be embodied on a computer readable medium.

While the present invention has been described at some length and with some particularity with respect to the several embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A computer-implemented method for detecting spinal fractures, comprising:
receiving three-dimensional image data including at least a portion of a spine of a subject;
identifying the spine of the subject in the three-dimensional image data;
defining a spline approximating a local curvature along the spine of the subject;
defining multiple volumes of interest (VOIs), each VOI containing at least a portion of a vertebra of the spine of the subject, wherein each VOI is defined relative to an adjacent segment of the spline; and
identifying a fracture in at least one of the VOIs.

2. The method of claim 1, wherein the spine is identified by a convolutional neural network (CNN) trained for segmenting the spine.

3. The method of claim 1, wherein for each VOI a corresponding center point is sampled at a location defined relative to the spline, wherein the center points of the VOIs are sampled at regular intervals along the spline.

4. The method of claim 3, wherein center points for adjacent VOIs are located so as to generate overlapping VOIs.

5. The method of claim 3, wherein each VOI is formed about the center point and is oriented based on a tangent of the spline adjacent the corresponding center point.

6. The method of claim 5, wherein after defining each VOI, each VOI is extracted and resampled from the three-dimensional image data to a target resolution.

7. The method of claim 1, wherein the fracture is identified by applying a convolutional neural network (CNN) to each VOI.

8. The method of claim 7, wherein the output of the CNN, when applied to each VOI, is a probability map identifying likely fractures within the corresponding VOI.

9. The method of claim 8, wherein the VOIs are defined such that adjacent VOIs overlap so as to generate multiple predictions for at least some equivalent voxels occurring in the multiple VOIs, wherein all predictions corresponding to a particular location are aggregated into a final probability map.

10. The method of claim 8, further comprising generating a final probability map from the probability maps associated with individual VOIs, the final probability map comprising a collation of the VOI probability maps into a coherent representation of the three-dimensional image data.

11. The method of claim 10, further comprising generating binary predictions based on the final probability map and filtering fracture candidates based on a relationship between a candidate location and the spine of the subject.

12. The method of claim 1, wherein the spline approximates a centerline of a spinal canal for the spine.

13. The method of claim 1, wherein a size for a first VOI is selected based on an adjacent first location along the spine, and wherein a size for a second VOI is selected based on an adjacent second location along the spine.

14. The method of claim 1, further comprising locating fractures identified in a representation of the three-dimensional image data and displaying identified fractures in the context of the spine of the subject.

15. An apparatus, comprising:
a memory that stores a plurality of instructions; and
a processor that couples to the memory and is configured to execute the plurality of instructions to:
receive three-dimensional image data including at least a portion of a spine of a subject;
identify the spine of the subject in the three-dimensional image data;
define a spline approximating a local curvature along the spine of the subject;
define multiple volumes of interest (VOIs), each VOI containing at least a portion of a vertebra of the spine of the subject, wherein each VOI is defined relative to an adjacent segment of the spline; and
identify a fracture in at least one of the VOIs.
